(19) United States
(12) Patent Application Publication
(10) Pub. No.: US 2009/0163409 A1
(43) Pub. Date: Jun. 25, 2009

(54) Trappin-2 (elafin) inhibits HIV

(76) Inventors: T. Blake Ball, Winnipeg (CA); Shehzad Iqbal, Winnipeg (CA); Francis Plummer, Winnipeg (CA)

Correspondence Address: ADE & COMPANY INC., 2157 Henderson Highway, WINNIPEG, MB, R2G1P9, CA

(21) Appl. No.: 11/914,392

(22) PCT Filed: May 16, 2006
(86) PCT No.: PCT/CA06/00790

§ 371 Date: Jul. 9, 2008

Related U.S. Application Data
(60) Provisional application No. 60/681,016, filed on May 16, 2005.

(51) Int. Cl. A61K 38/16; C07K 14/00; A61P 31/18

(52) U.S. Cl. 514/12; 530/324

(57) ABSTRACT

In sub-Saharan Africa, the vast majority of HIV transmission occurs through heterosexual contact, therefore, the initial site of HIV infection occurs within the genital tract. In a cohort of HIV-highly exposed sex workers we have identified a select group of individuals who epidemiologically and clinically appear to be HIV-resistant. Studies of these women indicate a strong correlation of HIV-specific immune responses within the genital tract to protection from infection. We hypothesized that a characteristic immune phenotype is present within the genital tract of the HIV-resistant women when compared to susceptible controls. To test this we used SELDI-TOF mass spectrometry to profile the proteome of genital tract secretions from the HIV-resistant women and found a number of potential biomarkers (differentially expressed proteins) which correlated to HIV-resistance. Purification and tandem mass spectrometry resulted in the identification of a particular biomarker, namely trappin-2 (elafin). This protein was tested for HIV inhibitory activity in vitro and found to be a potent inhibitor of T tropic viral infection.

### PRIOR APPLICATION INFORMATION

This application claims the benefit of U.S. Provisional Application 60/681,016 filed May 16, 2005.

### BACKGROUND OF THE INVENTION

HIV/AIDS currently affects over 40 million individuals, the vast majority of whom live in sub-Saharan Africa. Despite strong prevention campaigns and the introduction of antiretroviral treatments, the spread of this disease continues. In 2004, the United Nations reported a significant shift in the dynamics of this disease, reporting that for the first time, more women were infected than men, a trend which is expected to continue and grow in the coming years. Due to complicated social, economic and cultural norms in many of the nations affected by HIV, women are at a substantially higher risk of contracting the disease and have few options to help protect themselves in male dominated societies. As a potential response to this problem, the United Nations and other large health policy organizations have called for the development of vaginal microbicides that are protective against HIV. Use of an effective microbicide would provide women with a choice in protecting themselves independently of their partner's will. Importantly, modeling has also shown that even a partially effective microbicide would greatly reduce the transmission rates of HIV and have a substantial impact on the global spread of HIV.

In Nairobi, Kenya, a group of commercial sex workers has been the subject of intense study of the immunobiology and pathogenesis of HIV infection. As a highly exposed population and a key transmitter group of the disease, studies of these women have led to important advances in our understanding of the disease. Of particular interest in this cohort is the identification of a small subset of women who, despite repeated exposure, have remained HIV uninfected as determined by both PCR and serology. Termed HIV-resistant, some have remained HIV uninfected for 20 years of constant exposure to infected clients. Studies by our research group and others have shown evidence that their protection from HIV is immune-mediated with some genetic familiarity associated with resistance as well. As HIV is primarily transmitted through heterosexual contact in sub-Saharan Africa, the first site of contact between the host and the virus is the genital tract. Thus, studies of the mucosal immune barrier of the female genital tract is of great interest. However, because of the limitations of sensitivity in immune assays these studies are difficult to perform. We have employed a proteomics platform technology called SELDI-TOF (surface enhanced laser desorption ionization-time of flight) mass spectrometry to overcome these difficulties and characterize the immune proteome of the genital tract of HIV resistant women. We hypothesized that HIV-resistant women exhibit a characteristic protein profile when compared to susceptible controls and that specific biomarkers (or proteins having differential expression between groups) could be identified using this technology. In turn, these biomarkers may have a direct role in mediating protection from HIV. One such biomarker has been purified and identified as trappin-2 (formerly referred to as elafin). It is further shown that trappin-2 inhibits HIV in a potentially novel manner.

### Trappin-2 (Elafin):

Trappin-2, otherwise known as elafin, skin-derived anti-leucoproteinase (SKALP) or elastase-specific inhibitor (ESI) is a serine protease inhibitor. This protein was isolated initially in a variety of settings around the same time, hence, the multiple names; however, upon molecular characterization, it was shown to be the same protein. It is now a considered a member of the Trappin family of genes for which there is only one form in humans, trappin-2. Trappin proteins contain a characteristic WAP (whey acidic protein) domain (residues 72-117) which is a four-disulfide bond core peptide in the C-terminus of the protein. This C-terminus is thought to contain the protein's anti-protease active site. In addition, the N-terminus of Trappin-2 contains a transglutaminase substrate domain (residues 23-60) composed of a repeating consensus sequence (Gly-Gln-Asp-Pro-Val-Lys) GQDPVK. This domain is thought to confer its ability to form polymers and stick to members of the extracellular matrix of tissues.

Trappin-2 is a secreted protein found primarily at mucosal surfaces and is thought to be a potent tissue-bound inhibitor of inflammation, responsible for maintaining the epithelial integrity. The protein is 117 amino acids in length, which includes a 22 amino acid hydrophobic signal peptide. In its full-length form, the protein is 12.3 kDa in size. Cleavage of the signal peptide yields a 9.9 kDa mature form of the protein. A further cleaved product is the 6 kDa form which is comprised of the 57 aa residues from the C-term end of the protein and does not contain the transglutaminase domain. Trappin-2 is normally not expressed in the epidermis of skin but is expressed in inflammatory conditions such as psoriasis. Other sites of expression previously described include the oesophagus, pharynx, vagina, and oral epithelium. Specifically, production is thought to occur in stratified epithelial tissues, although some evidence exists for production by macrophages as well (in lung tissue). It has also been found in sputum and bronchoalveolar lavage fluid. Trappin-2 has been found to be a potent and specific inhibitor of a restricted set of proteases, specifically leukocyte elastase and leukocyte proteinase-3, both derived from neutrophils. In addition, it is a substrate for transglutaminases which mediate the covalent binding to extracellular matrix proteins.

Trappin-2 is a constitutively expressed protein; however it has been shown to be inducible in response to TNFα and IFNγ. The gene for Trappin-2 is approximately 2.3 kb long and is composed of three exons and two introns. A 5′ regulatory sequence for AP-1 binding is present, suggesting a regulatory pathway through NF-κB. Because of its anti-inflammatory activities, trappin-2 has been studied in the context of many diseases. Due to its robust nature (small size, resistance to extreme pH, heat and oxidation), it has been tested as an anti-inflammatory agent for a number conditions (ie. lung emphysema, cystic fibrosis, reperfusion injury from myocardial infarction). However, to date no role for trappin-2 in resistance and susceptibility to HIV infection has been suggested.

Secretory Leukocyte Inhibitor-I (SLP-1) and Confusion with Trappin-2:

SLP-I is a protease inhibitor and member of the superfamily of proteins called ALP (antileukoprotease) whose only other member is Trappin-2. SLP-I was first isolated from saliva in humans and found to have anti-HIV affects. Since then, SLP-I has been found in most mucosal derived secretions including vaginal mucosa and breast milk. Recently, a proposed mechanism of inhibition was delineated whereby SLP-I binds to surface annexin receptors on macrophage cells and prevents binding of macrophage tropic (also called R5) viruses to these cells. SLP-I has been shown to inhibit HIV infection of macrophage cells but does not prevent infection of T cells using T cell tropic (or X4) viruses.

Interestingly, SLP-I and Trappin-2 have become synonymous in the HIV literature as evidenced when using search engines such as PubMed to review the literature on both these proteins. However, upon closer inspection, no previous work has been done to characterize the effects of Trappin-2 on HIV. This is important since Trappin-2 and SLP-I are completely different proteins that share similar function but have significant differences. For example, Trappin-2 is a highly restricted protease inhibitor for specific enzymes whereas SLP-I has been shown to block a number of other proteases. In addition, there are differential regulatory patterns for each of the proteins.

Part of the confusion over the relation between SLP-I and Trappin-2 may have arisen due to their chromosomal location and sequence homology. Both Trappin-2 and SLP-I are located on chromosome 20 in the 20q12-13 region, side-by-side. In addition, the C-terminus of Trappin-2 shares 38% homology to the C-terminus of SLP-I. Though this is a highly conserved domain throughout many species, it is considered the active domain of both these proteins, yet shows different functional effects. In contrast to trappin-2, SLP-I does not contain a transglutaminase N-terminus domain in its protein structure. Trappin-2 is thought to have evolved through exon shuffling of SLP-I and the REST genes, a group of seminal-vesicle transglutaminase substrates. Both these genes share considerable homology in their exon structure and sequence homology within their noncoding intron regions as well. While SLP-I encodes for a C-terminus WAP motif, the N-terminus transglutaminase domain in the Trappin-2 protein is thought to have come from the REST gene. All three of these genes are co-localized to the same location on chromosome 20.

### SUMMARY OF THE INVENTION

According to a first aspect of the invention, there is provided a purified HIV inhibitory peptide comprising a peptide having at least 60% identity to SEQ ID NO: 1.

According to a second aspect of the invention, there is provided a method of inhibiting HIV infection comprising:

administering to an individual in need of such treatment an effective amount of a peptide having at least 60% identity to SEQ ID NO: 1.

According to a third aspect of the invention, there is provided use of a purified HIV inhibitory peptide comprising a peptide having at least 60% identity to SEQ ID NO: 1 for inhibiting HIV infection.

According to a fourth aspect of the invention, there is provided use of a purified HIV inhibitory peptide comprising a peptide having at least 60% identity to SEQ ID NO: 1 in manufacturing a pharmaceutical composition for inhibiting HIV infection.

### BRIEF DESCRIPTION OF THE DRAWINGS

FIG. 1. SELDI-TOF MS data analysis reveals a 6 kDa protein peak associated with HIV-resistance. (A) Representative protein mass spectra of CVL samples from HIV-uninfected (n=30), infected (n=161) and resistant (n=124) individuals. The 6.0 kDa protein of interest is highlighted. (B) Intensity plot of the average values for the 6.0 kDa protein peak in each individual for samples collected over multiple time points. HIV-resistant individuals showed a 2.1 fold increase in the 6 kDa peak compared to the HIV-uninfected control group (p=0.0001) and a 1.3 fold increase compared to HIV-infected (p=0.0007).

FIG. 2. Identification of the 6 kDa protein as trappin-2. The 6 kDa polypeptide was purified from CVL by anion exchange chromatography (flow-through) followed by reverse phase chromatography (30% acetonitrile, 0.1% trifluoroacetic acid). The enriched preparation was reduced, alkylated and resolved by SDS-PAGE. Gel-purified protein was digested with trypsin and unique fragments analyzed by tandem MS. A) An ion with m/z of 2347 was fragmented by collision induced dissociation (CID) and the MS/MS spectrum was submitted to Mascot search tool for identification. The ion was identified as a carbamidomethylated tryptic fragment of trappin-2 with probability based Mowse score of 56 (score>26 indicates identity or extensive homology). B) CID spectrum of the 2347 m/z. C) An ion with m/z of 1072 was fragmented by CID and the MS/MS spectrum was submitted to Mascot search tool for identification. The ion was identified as a carbamidomethylated tryptic fragment of trappin-2 with probability based Mowse score of 42 (score>16 indicates significant homology, score>21 indicates identity or extensive homology). D) CID spectrum for the ion with m/z of 1072. E) Amino acid sequence of the 6 kDa biomarker identified as trappin-2. Peptides directly identified by MS/MS are underlined. The sequence highlighted in red corresponds to trappin-2. The calculated MW of trappin-2 is 6007.20 Da. Considering four disulfide bridges in the polypeptide, the corrected MW is 5999.20 Da. The latter MW matched experimentally observed m/z values with high accuracy (see FIG. 1a). F) Four ions identified by MS/MS as tryptic fragments of trappin-2.

FIG. 3. Trappin-2 is significantly over-expressed in the HIV-resistant study group as determined by ELISA. A) As no specific Ab exists for the 6 kDa form of Trappin-2 we assayed for detection of both the 6 kDa and 9.9 kDa form of trappin-2 by an in-house ELISA. HIV-resistant individuals showed a 2.6 fold increase in trappin-2 expression compared to HIV-uninfected (p<0.01) and a 1.8 fold increase compared to HIV-infected (p<0.0004) individuals. B) A commercially available ELISA kit (HyCult Biotechnology) was used to quantify the 9.9 kDa form of trappin-2. HIV-resistant individuals showed a 3.3 fold increase in trappin-2 levels compared to HIV-uninfected (p<0.002) and a 1.5 fold increase compared to HIV-infected (p<0.01) individuals. There was no significant difference between the HIV-uninfected and HIV-infected individuals. All bars represent the median value; analysis was by Mann Whitney U test.

FIG. 4. Trappin-2 inhibits HIV viral replication in an in vitro system. A) We tested HIV HxBc2 in an established viral replication model system and measured the effect of pretreatment of recombinant human trappin-2 (6.0 kDa form) on cells in viral infection 15,19. Briefly, 1 or 5 ug/mL of trappin-2 was incubated with MT4 cells for 1 hour. Then equivalent amounts of virus (0.02 cpm/cell) were added to the cells in the presence of trappin-2 for 2 hours. Infected cells were washed and cultured in the absence of trappin-2. As a control, 10 uM of AZT was used. Viral production in supernatants was evaluated by RT activity after 6 days of culture. We show that pretreatment of trappin-2 for 1 hour on MT4 cells greatly reduced HIV viral replication in a dose dependent manner. B) The experiment was repeated with an additional assay of trappin-2 preincubated with cells and subsequently supplemented into the culture media (lane 6). Supernatants were harvested at Day 6 of infection and assayed for both RT activity and western blot analysis for HIV p24. Error bars are mean±SEM.

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

As used herein, “effective amount” refers to the administration of an amount of a given compound that achieves the desired effect. Regarding Trappin-2, a variant or bioactive fragment thereof, “effective amount” refers to an amount sufficient to inhibit or reduce HIV infection.

As used herein, “purified” does not require absolute purity but is instead intended as a relative definition. For example, purification of starting material or natural material to at least one order of magnitude, preferably two or three orders of magnitude is expressly contemplated as falling within the definition of “purified”.

As used herein, the term “isolated” requires that the material be removed from its original environment.

As used herein, “conservative substitution” refers to substitution of an amino acid with an amino acid that has similar properties such that one of skill in the art would anticipate or predict that the secondary structure and hydropathic nature of the polypeptide would be substantially unchanged.

As used herein, “variant” refers to allotypes known in the art that comprise one or more amino acid changes within a given sequence.

As used herein, “bioactive fragment” refers to a fragment of Trappin-2 or a variant or allotype or isoform thereof which retains HIV inhibitory activity.

Described herein is the use of purified, isolated or synthetic Trappin-2, variant thereof or a bioactive fragment thereof to inhibit, prevent or reduce the frequency or efficiency of human immunodeficiency virus infection. In a preferred embodiment, the HIV inhibitory peptide comprises or consists of or consists essentially of:

aqepvkgpvs tkpgscpiil ircamlnppn ircamlnppn rclkdtdcpg ikkccegscg macfvpq (SEQ ID NO: 1). As discussed below, this corresponds to the 6.6 kDa peptide.

In other embodiments, the HIV inhibitory peptide comprises or consists essentially of or consists of:

avtgvpvk gqdtvkgrvp fngqdpvkgq vsvkgdkvk aqepvkgpvs tkpgscpiil ircamlnppn ircamlnppn rclkdtdcpg ikkccegscg macfvpq (SEQ ID NO: 2). As discussed below, this corresponds to the full-length Trappin-2 peptide having had the signal sequence cleaved off. In a yet further embodiment, the HIV inhibitory peptide comprises or consists of or consists essentially of:
mrassflivv vfliagtlvl eaavtgvpvk gqdtvkgrvp fngqdpvkgq vsvkgdkvk aqepvkgpvs tkpgscpiil ircamlnppn ircamlnppn rclkdtdcpg ikkccegscg macfvpq (SEQ ID NO: 3).

While not wishing to be bound to a particular hypothesis, as discussed below, it is believed that the HIV inhibitory peptide blocks HIV binding to T cells, for example, by binding to T cells and occupying the HIV binding site.

It is of note that It is well known in the art that some modifications and changes can be made in the structure of a polypeptide without substantially altering the biological function of that peptide, to obtain a biologically equivalent polypeptide. In one aspect of the invention, the above-described peptides may include peptides that differ by conservative amino acid substitutions. The peptides of the present invention also extend to biologically equivalent peptides that differ by conservative amino acid substitutions. As used herein, the term “conserved amino acid substitutions” refers to the substitution of one amino acid for another at a given location in the peptide, where the substitution can be made without substantial loss of the relevant function, in this case, the folding of the epitope. In making such changes, substitutions of like amino acid residues can be made on the basis of relative similarity of side-chain substituents, for example, their size, charge, hydrophobicity, hydrophilicity, and the like, and such substitutions may be assayed for their effect on the function of the peptide by routine testing.

In some embodiments, conserved amino acid substitutions may be made where an amino acid residue is substituted for another having a similar hydrophilicity value (e.g., within a value of plus or minus 2.0), where the following may be an amino acid having a hydropathic index of about −1.6 such as Tyr (−1.3) or Pro (−1.6)s are assigned to amino acid residues (as detailed in U.S. Pat. No. 4,554,101, incorporated herein by reference): Arg (+3.0); Lys (+3.0); Asp (+3.0); Glu (+3.0); Ser (+0.3); Asn (+0.2); Gln (+0.2); Gly (0); Pro (−0.5); Thr (−0.4); Ala (−0.5); His (−0.5); Cys (−1.0); Met (−1.3); Val (−1.5); Leu (−1.8); Ile (−1.8); Tyr (−2.3); Phe (−2.5); and Trp (−3.4).

In alternative embodiments, conserved amino acid substitutions may be made where an amino acid residue is substituted for another having a similar hydropathic index (e.g., within a value of plus or minus 2.0). In such embodiments, each amino acid residue may be assigned a hydropathic index on the basis of its hydrophobicity and charge characteristics, as follows: Ile (+4.5); Val (+4.2); Len (+3.8); Phe (+2.8); Cys (+2.5); Met (+1.9); Ala (+1.8); Gly (−0.4); Thr (−0.7); Ser (−0.8); Trp (−0.9); Tyr (−1.3); Pro (−1.6); His (−3.2); Glu (−3.5); Gin (−3.5); Asp (−3.5); Asn (−3.5); Lys (−3.9); and Arg (−4.5).

In alternative embodiments, conserved amino acid substitutions may be made where an amino acid residue is substituted for another in the same class, where the amino acids are divided into non-polar, acidic, basic and neutral classes, as follows: non-polar: Ala, Val, Len, lie, Phe, Trp, Pro, Met; acidic: Asp, Glu; basic: Lys, Arg, His; neutral: Gly, Ser, Thr, Cys, Asn, Gln, Tyr.

Accordingly, in some embodiments, the HIV inhibitory peptide is derived from Trappin-2, that is, the HIV inhibitory peptide comprises a peptide that has at least 60% or at least 65% or at least 70% or at least 75% or at least 80% or at least 81%, or at least 82%, or at least 83% or at least 84% or at least 85% or at least 86% or at least 87% or at least 88% or at least 89% or at least 90% or at least 91% or at least 92% or at least 93% or at least 94% or at least 95% identity with SEQ ID NO: 1, for example, with amino acids 1-67 of SEQ ID NO. 1 and retains HIV inhibitory activity, as discussed below. As will be appreciated by one of skill in the art, in some embodiments, the HIV inhibitory peptide as described above may be genetically or chemically fused to a carrier protein. That is, the HIV inhibitory peptide may be crosslinked to a carrier protein or a nucleic acid molecule encoding the HIV inhibitory peptide may be inserted into an expression system such that the HIV inhibitory peptide is flanked at either its native N terminal or C terminal or both non-native amino acid residues.

It is of note that in addition to being an HIV inhibitory peptide, the above-described peptides are also immunotherapeutic agents in that they will modify the immune response to HIV or alter targeted cells of infection (which are immune cells) such that they are rendered impermeable or substantially impermeable to the virus and/or are no longer able to support viral replication.

In other embodiments, there is provided an expression system comprising a nucleic acid deduced from the amino acid sequence of any one of the above-described peptides, preferably SEQ ID NO: 1 or a variant or bioactive fragment thereof, operably linked to a suitable promoter. As will be appreciated by one of skill in the art, the suitable promoter may be for expression and recovery of the peptide, for example, in yeast, baculovirus or other suitable expression systems, or a strong inducible or constitutive host-specific promoter for construction of gene therapy or gene replacement vectors or suitable promoters for transient expression.

In yet other embodiments, the HIV inhibitory peptide as described herein may be fused to known peptide domains, for example, for targeting (signal domains or targeting domains) or purification (affinity domains) of the peptides. Such chimeric peptides are within the scope of the invention.

In other embodiments, the purified, isolated or synthesized peptide as described above is administered to an individual in need of such treatment for inhibiting or reducing human immunodeficiency virus infection.

In yet other embodiments, the purified, isolated or synthesized peptide as described above is used in the manufacture of a pharmaceutical composition for inhibiting or reducing human immunodeficiency virus infection. In a preferred embodiment, the peptide is the active agent in a composition arranged to be applied vaginally, as discussed herein.

In yet other embodiments, the purified, isolated or synthesized peptide as described above is used in the manufacture of a microbicide, as discussed herein. As will be appreciated by one of skill in the art, in these embodiments, one or more of the HIV inhibitory peptides may be combined with known microbicides effective against common vaginal tract pathogens. As discussed below, in these embodiments, the medicament comprising one or more of the HIV inhibitory peptides may be arranged for topical administration.

In some embodiments, one or more of the peptides described above may be combined with a pharmaceutically or pharmacologically acceptable carrier, excipient or diluent, either biodegradable or non-biodegradable. Exemplary examples of carriers include, but are by no means limited to, for example, poly(ethylene-vinyl acetate), copolymers of lactic acid and glycolic acid, poly(lactic acid), gelatin, collagen matrices, polysaccharides, poly(D,L lactide), poly(malic acid), poly(caprolactone), celluloses, albumin, starch, casein, dextran, polyesters, ethanol, mathacrylate, polyurethane, polyethylene, vinyl polymers, glycols, mixtures thereof and the like. Standard excipients include gelatin, casein, lecithin, gum acacia, cholesterol, tragacanth, stearic acid, benzalkonium chloride, calcium stearate, glyceryl monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, polyoxyethylene stearates, colloidol silicon dioxide, phosphates, sodium dodecylsulfate, carboxymethylcellulose calcium, carboxymethylcellulose sodium, methylcellulose, hydroxyethylcellulose, hydroxypropylcellulose, hydroxypropylmethycellulose phthalate, noncrystalline cellulose, magnesium aluminum silicate, triethanolamine, polyvinyl alcohol, polyvinylpyrrolidone, sugars and starches. See, for example, Remington: The Science and Practice of Pharmacy, 2000, Gennaro, A R ed., Eaton, Pa.: Mack Publishing Co.

As will be apparent to one knowledgeable in the art, specific carriers and carrier combinations known in the art may be selected based on their properties and release characteristics in view of the intended use. Specifically, the carrier may be pH-sensitive, thermo-sensitive, thermo-gelling, arranged for sustained release or a quick burst. In some embodiments, carriers of different classes may be used in combination for multiple effects, for example, a quick burst followed by sustained release.

In some embodiments, one or more of the HIV inhibitory peptides described above in any suitable form as described above may be combined with biological or synthetic targeting molecules, for example, site-specific binding proteins, antibodies, lectins or ligands, for targeting the HIV inhibitory peptides to a specific region or location.

In alternative embodiments, the HIV inhibitory peptides may be formulated for administration to a specific region of the body, for example, vaginally. That is, in these embodiments, the HIV inhibitory peptide is combined with a suitable carrier or excipient such that the medicament is arranged for vaginal administration. It is of note that methods for preparation of medicaments for vaginal application are well known in the art, see for example U.S. Pat. No. 6,432,440 which is incorporated herein by reference for this purpose.

In other embodiments, the HIV inhibitory peptide may be combined with other known treatments as a form of joint therapy. For example, the HIV inhibitory peptide may be combined with other anti-HIV compounds, for example, azidothymidine (AZT), lamivudine (3TC), dideoxyinosine (ddi), dideoxycytidine (ddc) and ritonavir, as well as other reverse transcriptase and protease inhibitors.

We describe here the discovery of a previously uncharacterized human protein that inhibits HIV infection in an in vitro model. Importantly, this protein was discovered in HIV resistant women with its elevated expression tightly associated with HIV-resistance. HIV-resistance in humans is a very rare phenomenon with only a handful of groups claiming to have identified and studied these individuals. The cohort we have characterized among the best characterized in the world. In addition, while mechanisms of HIV-resistance have been described (ie. the CCR5 32 deletion in Caucasian individuals) no known mechanism of resistance has been correlated to the women within this study group to date. Given the high exposure rates through heterosexual contact and the fact that once infected with HIV, it is impossible to clear, we hypothesized that these HIV resistant women are protected from infection at the genital mucosal barrier, thereby preventing infection from ever occurring. The discovery of a protein that is elevated in HIV-resistant women, expressed in the genital tract mucosa, and directly inhibits HIV-infection, lends strong support to our hypothesis. This is an important finding as no other inhibitor expressed at the genital tract level (ie. beta-chemokines or SDF-1, SLP-1, lactoferrin etc. . . . ) has ever correlated with HIV-resistance in humans. Interestingly, the mechanism by which trappin-2 seems to be inhibiting HIV-infection is novel when compared to other known inhibitors, including the related protein SLP-1, as discussed below.

As discussed herein, the protein can be used as a preventative measure/therapeutic intervention for HIV infection. As discussed herein, the peptide may be used for example either as an active ingredient in a microbicide or as an adjuvant or component of a therapeutic or preventative mucosal based vaccine. The United Nations has modeled the effective distribution and use of a hypothetical microbicide and shown that this preventative measure would save millions of lives every year.

As the initial site of infection with HIV in this cohort is the genital tract, it seems logical that mechanisms involved in mediating protection would be evident at the genital mucosa. To investigate potential mechanisms of protection against HIV we employed a novel approach to screen the proteome of genital tract secretions from HIV-resistant sex-workers and comparison groups. The protein content of cervical lavage (CVL) samples was analyzed using surface-enhanced laser desorption/ionization time-of-flight mass spectrometry (SELDI-TOF MS). This approach uses ProteinChip Arrays capable of specifically capturing proteins based on their chemical properties onto chromatographic surfaces. Bound proteins are then detected and characterized by linear time-of-flight mass spectrometry. Protein(s) of interest are then purified, sequenced and identified by tandem mass spectrometry. This strategy has advantages for study of mucosal material with limited sample volume in that it can quantitatively detect multiple proteins in a complex sample, identify unknown or unsuspected proteins, has high sample through-put and is highly sensitive (8, 9).

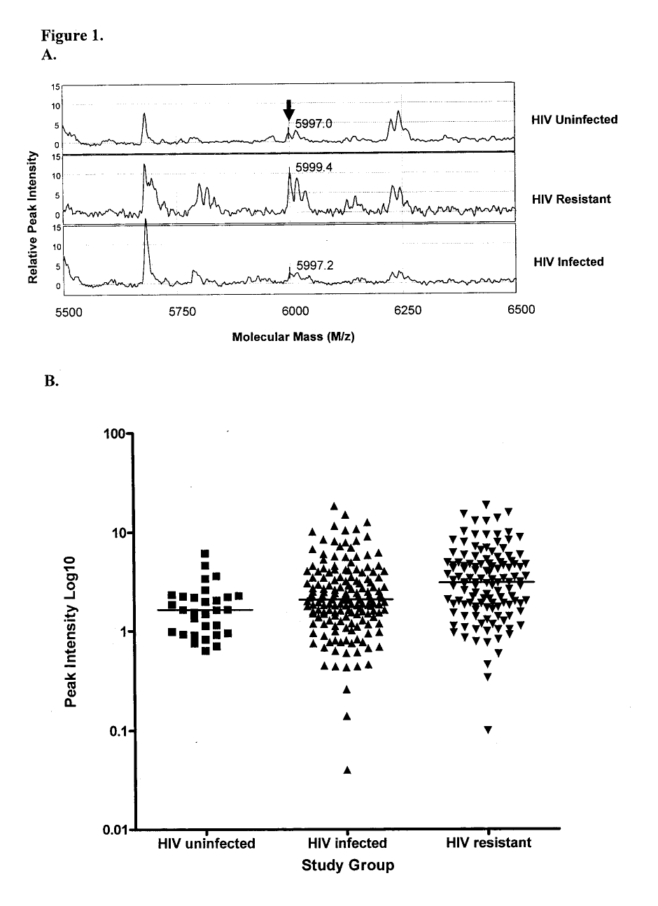

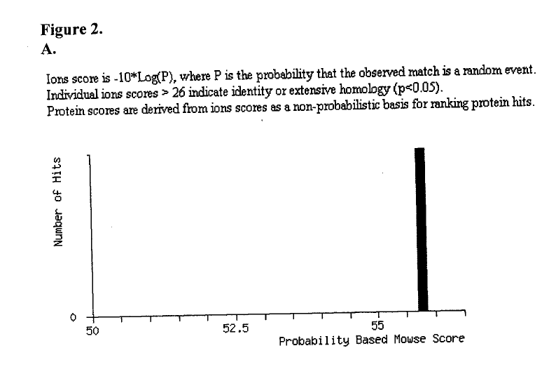

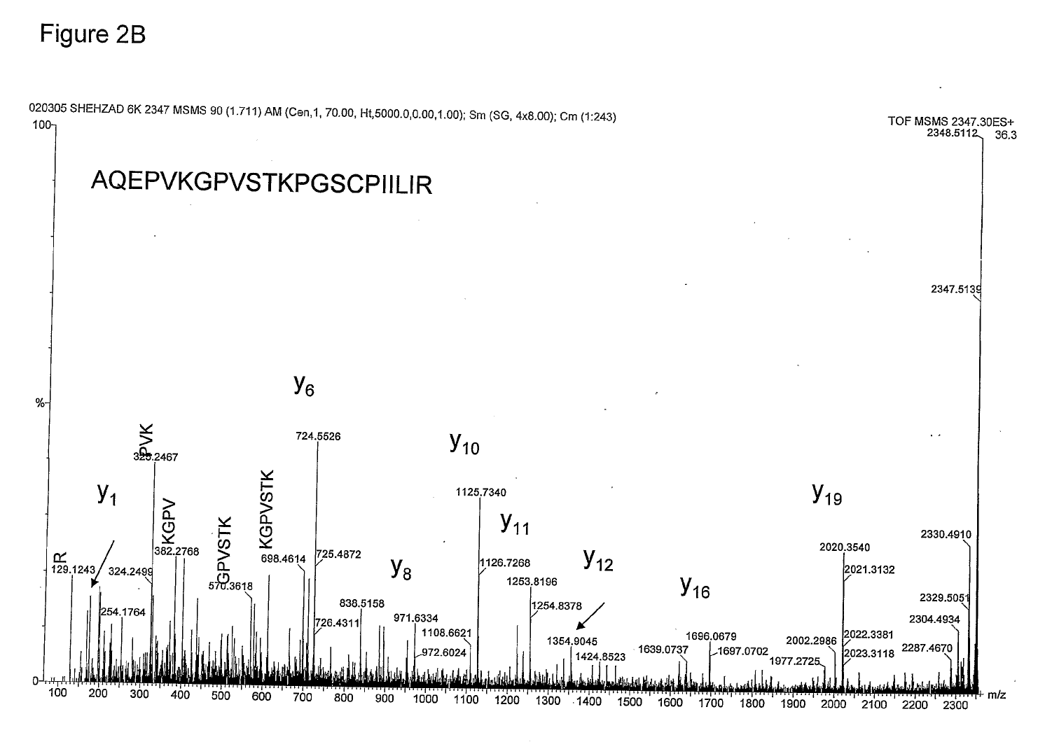

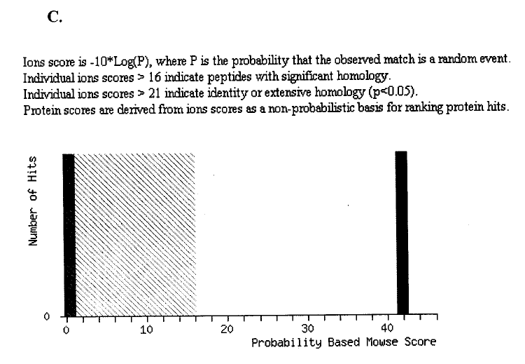

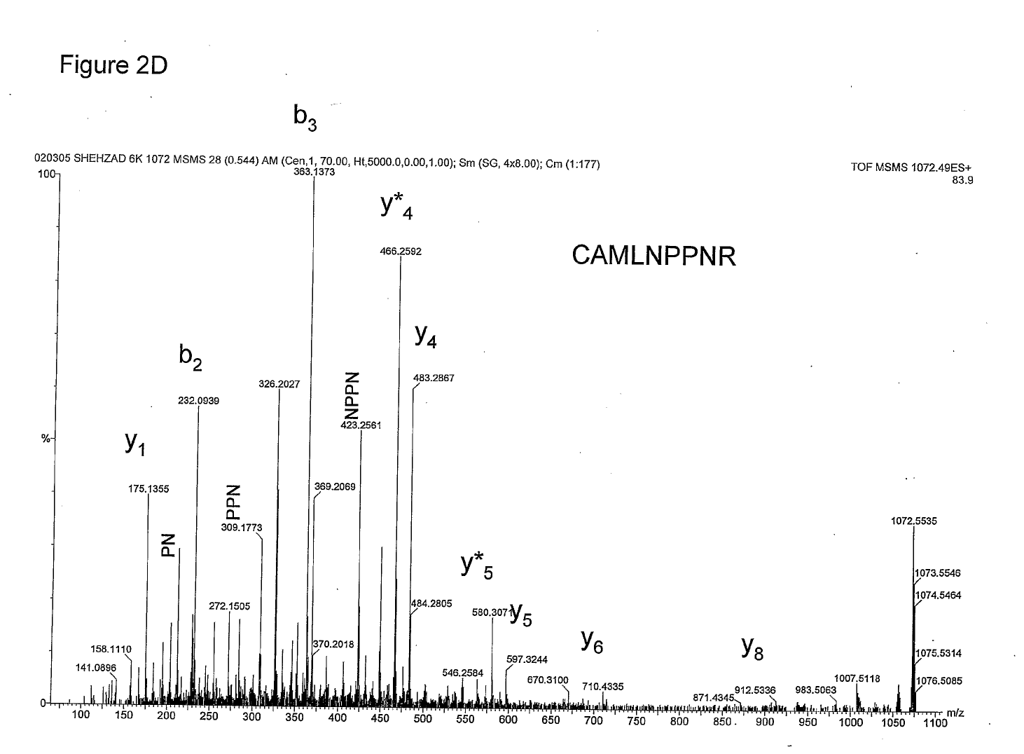

The genital tract proteome of HIV-resistant sex workers was compared to HIV uninfected women from the same sex worker cohort, with similar socioeconomic and genetic backgrounds, but who did not meet our epidemiologic definition of resistance. HIV-infected sex-workers from the same cohort were also studied. Assay optimization showed that the CM10 ProteinChip Array (cation exchanger) bound the greatest diversity of proteins from genital tract secretions and was chosen for subsequent analysis. A total of 579 CVL samples collected at multiple time points from 330 individuals were analyzed by SELDI-TOF MS. Spectral analysis revealed a number of proteins that were differentially expressed in the HIV-resistant sex-worker group compared to controls. In particular, one 6 kDa protein was over expressed in the genital tract secretions of the HIV-resistant study group compared to HIV-uninfected (p<0.001) and HIV-infected individuals (p<0.000001) (FIG. 1). This 6 kDa protein was purified from CVL samples and identified using tandem MS as the 6.0 kDa form of trappin-2, also known as human elafin/SKALP (skin-derived anti-leukoproteinase) (FIG. 2).

Trappin-2 is a member of the antileukoproteinase superfamily (ALS) of protease inhibitors. It has been largely studied in the context of lung disease, where its' antiprotease activity is thought to play a role in maintaining tissue integrity 10. Trappin-2 has also been shown to have anti-bacterial and anti-inflammatory properties (10,11). The protein is produced at mucosal surfaces by epithelial cells and possibly macrophages in three major forms: a 12.3 kDa cell associated form, as well as two secreted forms, the 9.9 kDa pre-trappin-2 and a 6 kDa active form. Trappin-2 also shares partial homology with secretory leukocyte protease inhibitor (SLPI), the other member of the ALS family, previously shown to inhibit the infection of macrophages by HIV (12). Despite their homology, these two proteins exhibit significant structural and functional differences (10), as discussed above.

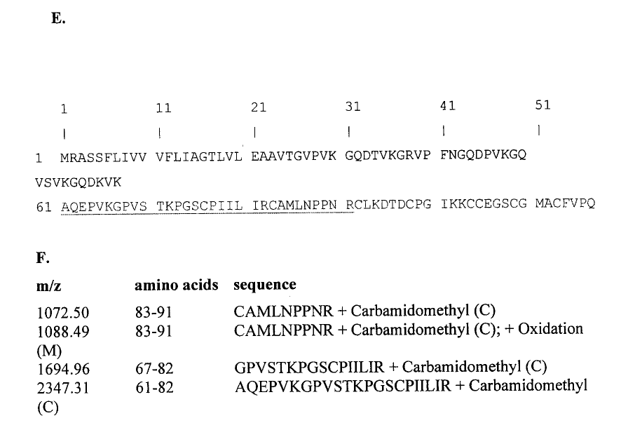

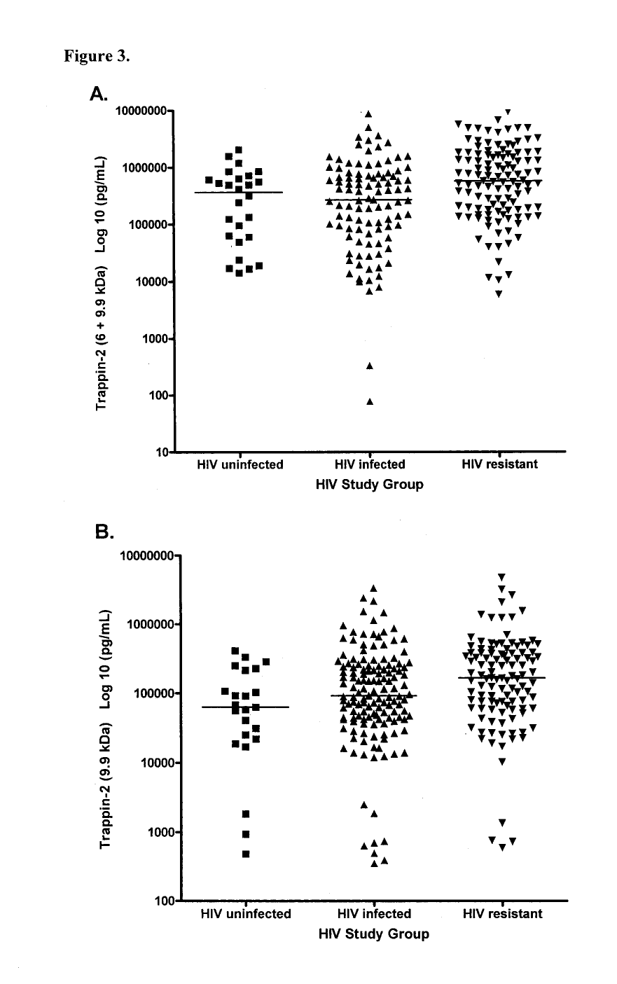

An enzyme immunoassay for trappin-2 was used to confirm the elevated levels of trappin-2 in the genital tract of HIV-resistant women. As no specific antibody to the 6.0 kDa form of the protein is available, an antibody specific for both the 6.0 and 9.9 kDa forms of the protein and an antibody specific to the 9.9 kDa form was used. Over expression of trappin-2 in the HIV-resistant study group compared to HIV-uninfected and HIV-infected individuals was confirmed (p=0.01 and p=0.004 respectively, FIG. 3a). Interestingly, the 9.9 kDa form of trappin-2 was elevated in the HIV-resistant population compared to HIV-uninfected and HIV-infected individuals (p=0.002 and p=0.01 respectively, FIG. 3b). The MS intensity values of the 6 kDa trappin-2 peak correlated strongly with the values obtained via the ELISA (p=0.000001), but showed no correlation with the 9.9 kDa ELISA data, illustrating the specificity of our MS approach. Finally, since trappin-2 has significant homology with SLPI, we assessed SLPI levels in genital tract secretions by ELISA. No differences in SLPI levels were found between study groups. Multivariate analysis of trappin-2 levels showed no relationship between levels of this protein with potential confounding variables such as menses, contraceptive use and concomitant infections.

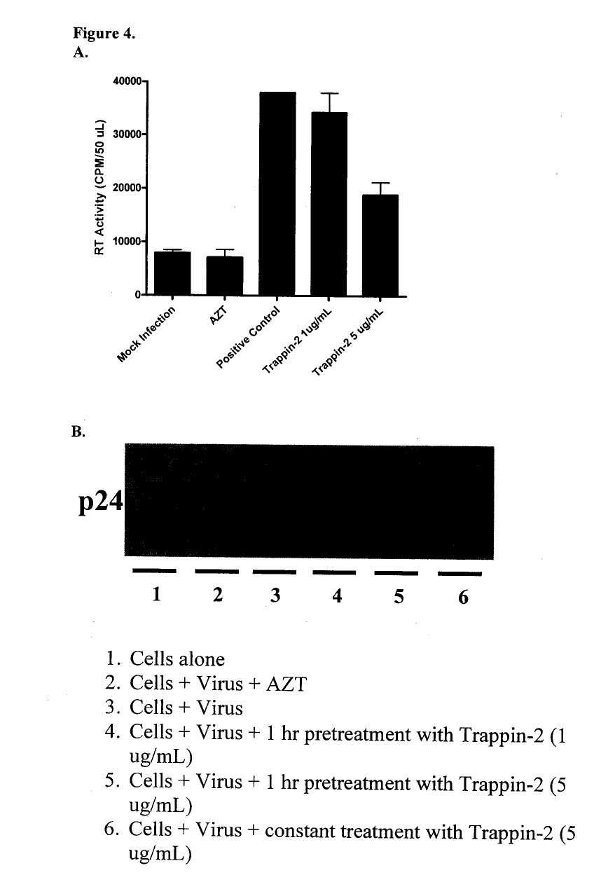

Finally, the ability of trappin-2 (6.0 kDa form) to inhibit HIV infection in an established HIV infection model was examined. A highly infectable T cell line (MT4) was used to assay the effect of trappin-2 inhibition on HIV HxBc2 replication 13,14,15. HIV inhibition experiments were performed by preincubating trappin-2 with either the virus, or target cells. A one-hour pre-exposure of cells to trappin-2 at physiological concentrations significantly reduced virus replication (FIG. 4). These data suggest that HIV entry was being prevented, since the simultaneous addition of trappin-2 and virus to target cells resulted in significantly less inhibition. When trappin-2 was added back into the culture system after the initial one-hour preincubation, there was an even more dramatic decrease in viral replication (FIG. 4b). These data suggest that even a 2-3 fold increase in trappin-2 concentration at mucosal surfaces, such as that noted in the HIV-resistant sex workers, could potentially have significant in vivo effects. In our in vitro system a 5 fold increase in trappin-2 (FIG. 4b) had significant effects on HIV replication. Trappin-2 has previously been reported to have immunomodulatory effects and thus its influence on immune regulation is of interest. To determine if the HIV inhibitory effects of trappin-2 are the result of induction of anti-viral cytokines such as IFN-γ, cytokine responses of MT4 cells after treatment with trappin-2 were measured. No induction of cytokines was detected.

Although other inhibitors of HIV have been found at the mucosal barrier such as SLPI and the chemokine SDF-1, they have not been shown to correlate with natural immunity to HIV 12 16. Recently, we described elevated levels of the β-chemokine RANTES within the genital tract of HIV-resistant women 17. Here we have identified a new inhibitor of HIV within the genital tract of these women, using a powerful new technology. In conjunction with other naturally expressed inhibitors of HIV such as RANTES, trappin-2 may be forming an effective barrier within the genital tract of the HIV-resistant women, preventing infection. Given the strong correlation of trappin-2 with resistance, we believe that this protein plays an important role in preventing HIV infection. When we stratified women who had greater than the mean trappin-2 levels±1 SD found in the HIV-uninfected group, we were able to show that individuals who had trappin-2 levels greater than this amount showed a significantly reduced likelihood of being HIV infected, with a protective odds ratio (OR) of 5.9 (CI95% 1.9-24.4, p<0.006) towards the HIV-resistant group based on the MS data (only 4/30 HIV-uninfected, or susceptible women had trappin-2 levels over this threshold, compared to 65/114 of the HIV-resistant women), or an OR of 4.5 (CI95% 1.24-24.7, p<0.002) based upon the ELISA data (3/26 susceptible vs. 73/116 resistant). Of particular interest is the activity of trappin-2 against an X4 type virus. This is unexpected, as current models of HIV pathogenesis suggests that R5 type viruses are responsible for the majority of sexual transmission (18). While elevated levels of chemokines binding CCR5, such as RANTES, may play a role in protection against sexual acquisition of HIV, the strong association of HIV-resistance to elevated trappin-2 implies that ideal microbicides should be effective against both X4 and R5HIV viruses. Clearly, further studies are needed to elucidate the anti-viral mechanism(s) by which trappin-2 inhibits HIV, and its ability to inhibit across a variety of HIV strains and clades. However, the fact that resistant sex-workers demonstrate reduced susceptibility to multiple HIV clades in Kenya, including A-D and recombinants, implies that this protein has broad activity. In conclusion, we have identified an innate immune protein, trappin-2, which is significantly elevated in the genital tract of HIV-resistant sex-workers, and has potent anti-HIV activity at physiologic concentrations. Trappin-2 may play an important role in mediating natural immunity to HIV infection and may have potential as an HIV microbicide.

While the preferred embodiments of the invention have been described above, it will be recognized and understood that various modifications may be made therein, and the appended claims are intended to cover all such modifications which may fall within the spirit and scope of the invention.

### REFERENCES

1. Kaul, R. et al. HIV-1-specific mucosal CD8+ lymphocyte responses in the cervix of HIV-1-resistant prostitutes in Nairobi. J Immunol 164, 1602-11 (2000).
2. Kaul, R. et al. HIV-1-specific mucosal IgA in a cohort of HIV-1-resistant Kenyan sex workers. Aids 13, 23-9 (1999).
3. MacDonald, K. S. et al. Human leucocyte antigen supertypes and immune susceptibility to HIV-1, implications for vaccine design. Immunol Lett 79, 151-7 (2001).
4. Quinn, T. C. & Overbaugh, J. HIV/AIDS in women: an expanding epidemic. Science 308, 1582-3 (2005).
5. Fowke, K. R. et al. Resistance to HIV-1 infection among persistently seronegative prostitutes in Nairobi, Kenya. Lancet 348, 1347-51 (1996).
6. Devito, C. et al. Mucosal and plasma IgA from HIV-exposed seronegative individuals neutralize a primary HIV-1 isolate. Aids 14, 1917-20 (2000).
7. Devito, C. et al. Mucosal and plasma IgA from HIV-1-exposed uninfected individuals inhibit HIV-1 transcytosis across human epithelial cells. J Immunol 165, 5170-6 (2000).
8. Hutchens, T. W. & Yip, T.-T. New desorption strategies for the mass spectrometric analysis of macromolecules. Rapid Communications in Mass Spectrometry 7, 576-580 (1993).
9. Fung, E. T., Thulasiraman, V., Weinberger, S. R. & Dalmasso, E. A. Protein biochips for differential profiling. Curr Opin Biotechnol 12, 65-9 (2001).
10. Sallenave, J. M. The role of secretory leukocyte proteinase inhibitor and elafin (elastase-specific inhibitor/skin-derived antileukoprotease) as alarm antiproteinases in inflammatory lung disease. Respir Res 1, 87-92 (2000).
11. Simpson, A. J., Maxwell, A. I., Govan, J. R., Haslett, C. & Sallenave, J. M. Elafin (elastase-specific inhibitor) has anti-microbial activity against gram-positive and gram-negative respiratory pathogens. FEBS Lett 452, 309-13 (1999).
12. McNeely, T. B. et al. Secretory leukocyte protease inhibitor: a human saliva protein exhibiting anti-human immunodeficiency virus 1 activity in vitro. J Clin Invest 96, 456-64 (1995).
13. Harada, S., Koyanagi, Y. & Yamamoto, N. Infection of HTLV-III/LAV in HTLV-1-carrying cells MT-2 and MT-4 and application in a plaque assay. Science 229, 563-6 (1985).
14. Ratner, L. et al. Complete nucleotide sequence of the AIDS virus, HTLV-III. Nature 313, 277-84 (1985).
15. Lavallee, C. et al. Requirement of the Pr55gag precursor for incorporation of the Vpr product into human immunodeficiency virus type I viral particles. J Virol 68, 1926-34 (1994).
16. Agace, W. W. et al. Constitutive expression of stromal derived factor-1 by mucosal epithelia and its role in HIV transmission and propagation. Curr Biol 10, 325-8 (2000).
17. Iqbal, S. M. et al. Elevated T cell counts and RANTES expression in the genital mucosa of HIV-1-resistant Kenyan commercial sex workers. J Infect Dis 192, 728-38 (2005).
18. Meng, G. et al. Primary intestinal epithelial cells selectively transfer R5HIV-1 to CCR5+ cells. Nat Med 8, 150-6 (2002).
19. Yao, X. J. et al. Vpr stimulates viral expression and induces cell killing in human immunodeficiency virus type 1-infected dividing Jurkat T cells. J Virol 72, 4686-93 (1998).

1. Use of a purified HIV inhibitory peptide comprising a peptide having at least 60% identity to SEQ ID NO: 1 in manufacturing a pharmaceutical composition for inhibiting HIV infection.

2. The use according to claim 1 wherein the pharmaceutical composition is arranged for topical administration.

3. The use according to claim 1 wherein the pharmaceutical composition is arranged for vaginal administration.

4. A purified HIV inhibitory peptide comprising a peptide having at least 60% identity to SEQ ID NO: 1.

5. A method of inhibiting HIV infection comprising:
administering to an individual in need of such treatment an effective amount of a peptide having at least 60% identity to SEQ ID NO: 1.

6. Use of a purified HIV inhibitory peptide comprising a peptide having at least 60% identity to SEQ ID NO: 1 for inhibiting HIV infection.
